(19) United States
(12) Patent Application Publication
(10) Pub. No.: US 2026/0208141 A1
(43) Pub. Date: Jul. 23, 2026

(54) SYSTEM AND METHOD FOR ALKYLATION OF HYDROCARBONS

(71) Applicant: Valero Services, Inc., San Antonio, TX (US)

(72) Inventors: Jill Burns, Boerne, TX (US); Zachary Macdonald, Corpus Christi, TX (US); Alexandria Jenkins, Corpus Christi, TX (US)

(73) Assignee: Valero Services, Inc., San Antonio, TX (US)

(21) Appl. No.: 19/451,072

(22) Filed: Jan. 16, 2026

Related U.S. Application Data
(60) Provisional application No. 63/748,791, filed on Jan. 23, 2025.

(51) Int. Cl. B01J 19/00; B01J 14/00; C07C 2/62

(52) U.S. Cl. CPC B01J 19/0033; B01J 14/00; C07C 2/62; B01J 2219/00056; B01J 2219/00164; B01J 2219/00198; B01J 2219/002; B01J 2219/0022; B01J 2219/00231; B01J 2219/00238; B01J 2219/00261; C07C 2527/1206

(57) ABSTRACT

Provided is are methods and systems to operate a alkylation process, the method includes receiving, by one or more processors and from one or more sensors, measurements indicative of one or more operating parameters of an alkylation process; determining, by the one or more processors, a difference between the one or more operating parameters of the alkylation process and one or more target operating parameters of the alkylation process; determining, by the one or more processors, a factor associated with one or more of the one or more operating parameters, wherein the factor is based on the difference; and providing, by the one or more processors, an indication of the factor to an operator of the alkylation process.

### CROSS-REFERENCE TO RELATED APPLICATION(S)

This application claims the benefit of priority under 35 U.S.C. § 119(e) to U.S. Provisional Patent Application No. 63/748,791, filed on Jan. 23, 2025 and entitled “System and Method for Alkylation of Hydrocarbons,” the contents of which is incorporated herein by reference in its entirety.

### TECHNICAL FIELD

The present invention relates generally to the field of alkylation of hydrocarbons and specifically to improving alkylation systems utilizing acid catalyst.

### BACKGROUND

The alkylation process in petroleum refining is a chemical reaction that combines light iso-paraffins, such as isobutane (IC4), with olefins (e.g., propylene and butylene) to produce high-octane gasoline components known as alkylates. This process is crucial for enhancing the octane rating of gasoline, making it more efficient for modern engines. The reaction may be catalyzed by strong acids, typically sulfuric acid (H₂SO₄) or hydrofluoric acid (HF), which facilitate the formation of higher molecular weight iso-paraffins through ionic chain reactions.

For sulfuric acid alkylation, the reaction occurs at around 40° F., while hydrofluoric acid alkylation operates at approximately 100° F. The acid catalyst is mixed with the hydrocarbon feedstock in a reactor, where it promotes the reaction between the isobutane and olefins. Several parameters impact the effectiveness of the alkylation reaction, including the relative concentration of the components and catalyst. High acid strength is required for optimal performance of the alkylation reaction. Therefore, after the reaction, the mixture is sent to a settler where the hydrocarbons are separated from the acid. The acid is recycled back into the reactor for further use as a catalyst, and a partial stream of the acid is bypassed around the reactor and routed to an acid regeneration unit. Hydrofluoric acid (HF) is utilized in many modern refineries because it can be regenerated within the process, whereas sulfuric acid must be regenerated wholly external to the process.

Alkylation processes must be monitored to ensure economic and efficient performance.

### SUMMARY OF THE INVENTION

Provided herein are systems and methods of improving alkylation of hydrocarbons. The disclosure includes computer-implemented monitoring and control of an alkylation process.

In a first aspect, which may be combined with any other aspect or embodiment herein, a method of operating an alkylation process includes: receiving, by one or more processors and from one or more sensors, measurements indicative of one or more operating parameters of an alkylation process; determining, by the one or more processors, a difference between the one or more operating parameters of the alkylation process and one or more target operating parameters of the alkylation process; determining, by the one or more processors, a factor associated with one or more of the one or more operating parameters, wherein the factor is based on the difference; and providing, by the one or more processors, an indication of the factor to an operator of the alkylation process. In some embodiments, the method includes adjusting an operating parameter of the alkylation process in response to the factor reaching a predetermined value.

In some embodiments, the factor is a composite value determined according to two or more operating parameters. In some embodiments, the method also includes changing the position of a valve, a flow rate of isobutane, a flow rate of catalyst, a flow rate of olefin, or an operating temperature associated with the alkylation process in response to the factor. In some embodiments, the one or more operating parameters of the alkylation process include at least one of: an acid purity; an isobutane recycle purity; a ratio of a feed rate of isobutane to a feed rate of olefin; a feed rate of isobutane to a catalyst regenerator; a ratio of a feed rate of acid catalyst to a feed rate of hydrocarbon; an amount of fresh acid inventory; a feed rate of sulfur; a settler acid level; an alkylation reaction temperature; an acid boot valve position; a boiling end point of an alkylate; a mass of residue in an alkylate; a ratio of a flow rate of alkylate to a feed rate of olefin; an alkylate color; or a debutanizer boot level.

In a second aspect, which can be combined with any aspect or embodiment provided herein, a system for operating and/or monitoring an alkylation process includes: a control circuit comprising one or more processors and memory structured to store instructions that, when executed by the one or more processors, cause the control circuit to: receive, by one or more processors and from one or more sensors, measurements indicative of one or more operating parameters of an alkylation process; determine, by the one or more processors, a difference between the one or more operating parameters of the alkylation process and one or more target operating parameters of the alkylation process; determine, by the one or more processors, a factor associated with one or more of the one or more operating parameters, wherein the factor is based on the difference; and provide, by the one or more processors, an indication of the factor to an operator of the alkylation process. In some embodiments, the system is configured to adjust an operating parameter of the alkylation process in response to the factor reaching a predetermined value.

In some embodiments, the factor is a composite value determined according to two or more operating parameters. In some embodiments, the control circuit is configured to change the position of a valve, a flow rate of isobutane, a flow rate of catalyst, a flow rate of olefin, or an operating temperature associated with the alkylation process in response to the factor.

In a third aspect, presented herein is a method of alkylating an olefin in the presence of an acid catalyst, the method including: receiving, by one or more processors and from one or more sensors, measurements indicative of two or more operating parameters of an alkylation system; determining, by the one or more processors, a difference between the two or more operating parameters of the alkylation system and corresponding target operating parameters of the alkylation system; determining, by the one or more processors, a factor associated with two or more of the one or more operating parameters, wherein the factor is based on the difference of the two or more factors and the corresponding target operating parameters; and changing the position of a valve, a flow rate of isobutane, a flow rate of catalyst, a flow rate of olefin, or an operating temperature associated with the alkylation system in response to the factor, wherein the one or more operating parameters of the alkylation system include at least two or more of: an acid purity; an isobutane recycle purity; a ratio of a feed rate of isobutane to a feed rate of olefin; a feed rate of isobutane to a catalyst regenerator; a ratio of a feed rate of acid catalyst to a feed rate of hydrocarbon; an amount of fresh acid inventory; a feed rate of sulfur; a settler acid level; an alkylation reaction temperature; an acid boot valve position; a boiling end point of an alkylate; a mass of residue in an alkylate; a ratio of flow rate of alkylate to a feed rate of olefin; an alkylate color; or a debutanizer boot level.

In some embodiments, the method includes displaying, by the one or more processors, the factor to an operator of the alkylation system. In some embodiments, the receiving and determining steps are repeated periodically such that the factor is periodically updated. In some embodiments, the factor is updated every 5 minutes or less. In some embodiments, the changing step includes stopping a flow of olefin or stopping a flow of acid feed to the alkylation system when the factor exceeds a predetermined factor.

Both the foregoing summary and the following brief description of the drawings and detailed description are exemplary and explanatory. They are intended to provide further details of the invention, but are not to be construed as limiting. Other objects, advantages, and novel features will be readily apparent to those skilled in the art from the following detailed description of the invention.

### BRIEF DESCRIPTION OF THE DRAWINGS

FIG. 1 shows a schematic of a simplified alkylation system.

FIG. 2 is a flowchart of a method of operating an alkylation process according to an embodiment.

FIG. 3 is a schematic of a system for operating an alkylation process according to an embodiment.

### DETAILED DESCRIPTION

### Definitions

The following definitions are provided for clarity and are not intended to be limiting, except where explicitly recited in the claims.

As used herein, the terms “operating parameter,” “operating property,” and “monitored property” are used interchangeably and refer to any measurable or inferable quantity associated with an alkylation system that can be used to characterize its operation. Examples include, without limitation, process conditions (such as temperatures, pressures, flow rates, and liquid levels), composition-related properties (such as acid purity, sulfur content, and isoalkane purity), ratio-type parameters (such as isoalkane-to-olefin ratios and acid-to-hydrocarbon ratios), and product quality metrics (such as alkylate boiling end point, residue, and color).

As used herein, a “target operating parameter,” “target value,” or “target range” refers to a desired value or range of values associated with an operating parameter. A target may be a single setpoint, a continuous range, a time-dependent profile, or a value or range that is adjusted automatically or manually based on operating conditions. Differences between measured operating parameters and their corresponding targets are used to determine one or more factors as described herein.

As used herein, the terms “control circuit,” “control system,” and “monitoring system” refer to one or more devices including processors, memory, and associated hardware and/or software configured to perform the functions described herein. These terms are intended to encompass, without limitation, programmable logic controllers (PLCs), distributed control systems (DCS), embedded controllers, general-purpose computers, cloud-hosted servers, and combinations thereof, whether implemented as a single unit or as multiple distributed components communicating over one or more networks. Such control systems may be local or remote to the alkylation system.

As used herein, the term “sensor” refers broadly to any device or system that provides data indicative of an operating parameter, including but not limited to field instruments (e.g., flow meters, temperature sensors, pressure sensors, level sensors), composition analyzers, online analyzers (e.g., gas chromatographs, spectroscopic analyzers, density meters), laboratory instruments, and virtual or soft sensors that estimate a parameter based on models or correlations. The term “measurement” includes both direct readings from a sensor and values inferred, estimated, or calculated from such readings.

As used herein, “displaying” or “providing an indication” of a value, such as the factor, includes presenting the value in any human- or machine-readable form, such as on a graphical user interface, by changing a color or status indicator, by generating an audible or visual alarm, by transmitting a message over a network (for example, by email, text, or control signal), or by logging the value to a database or other storage medium for later review.

As used herein, the terms “automatic” and “automatically” refer to actions performed by the control circuit, control system, or monitoring system without requiring contemporaneous human intervention. An operator may configure parameters, thresholds, or operating modes in advance, but the real-time or near real-time execution of actions based on the factor can proceed automatically according to the programmed logic.

As used herein, the terms “real-time” and “near real-time” refer to processing and response that occur sufficiently quickly relative to the dynamics of the alkylation process to be useful for monitoring and control. Depending on the parameter, real-time or near real-time operation may correspond to update intervals on the order of seconds, tens of seconds, or minutes.

As used herein, the terms “operating parameter,” “operating property,” and “monitored property” are used interchangeably and refer to any measurable or inferable quantity associated with an alkylation system that can be used to characterize its operation. Examples include, without limitation, process conditions (such as temperatures, pressures, flow rates, and levels), composition-related properties (such as acid purity, sulfur content, and isoalkane purity), ratio-type parameters (such as isoalkane-to-olefin ratios and acid-to-hydrocarbon ratios), and product quality metrics (such as alkylate boiling end point, residue, and color). The term “factor” refers to any derived value as described above that is based at least in part on one or more such operating parameters and is indicative of an operational status of the alkylation system.

As used herein, the term “composite value” refers to a factor that is determined according to two or more operating parameters, whether by summation, weighted combination, nonlinear combination, or another form of aggregation. A composite value may be determined directly from the operating parameters or from differences between the operating parameters and their respective target values. A composite value may represent an overall index of process status or may represent a more specific index, such as an acid health index, a reaction severity index, or a product quality index.

### Overview

Alkylation is a refining process used to enhance the value of crude oil. In this process, smaller olefin molecules, such as propylene or butylene, react with a branched alkane like isobutane to produce higher-value alkylation products. These products are alkyls with longer chain lengths than the original reactants. For example, propene can react with isobutane to form 2,2-dimethylpentane, as shown in the reaction scheme below:

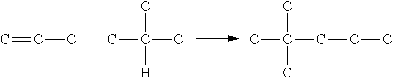

The alkylation reaction feed is flexible, requiring only an olefinic reactant and an isoalkane reactant. Thus, the products can encompass a wide range of hydrocarbon molecules. The isoalkane to olefin feed ratio heavily favors the isoalkane reactant. For example, the ratio of isoalkane to olefin feed rate may be about 4:1, 5:1, 6:1, 7:1, 8:1, 9:1, or 10:1 on a mass or molar basis. In some specific implementations, the ratio of isoalkane to olefin feed is at least 6:1 on a mass basis.

The alkylation reaction may be catalyzed by the presence of an acid. For example, in some alkylation processes, strong acids such as sulfuric acid (H₂SO₄) is used. In other processes, hydrofluoric acid (HF) is used as the catalyst. The acid catalyst protonates the olefin, creating a highly reactive carbocation intermediate that can then readily react with the isoalkane reactant to form a larger branched alkane product. For example, in some alkylation processes, H₂SO₄ or HF is used to protonate butylene to form a butyl cation intermediate, which can react with isobutane to form branched octane as a product.

Although many of the examples described herein refer to hydrofluoric acid (HF) or sulfuric acid (H₂SO₄) alkylation units, the methods and systems are not limited to these catalysts. In some embodiments, the alkylation process uses another strong acid catalyst, such as a mixed acid system, an ionic liquid acid catalyst, a solid acid catalyst used in conjunction with an acid regeneration or rejuvenation subsystem, or combinations thereof. The operating parameters monitored and used to determine the factor may be adapted to the particular catalyst and unit configuration, but may include analogous parameters such as catalyst activity indicators, catalyst regeneration status, and product quality metrics. Accordingly, the factor-based monitoring and control techniques described herein may be applied to a wide variety of acid-catalyzed alkylation processes.

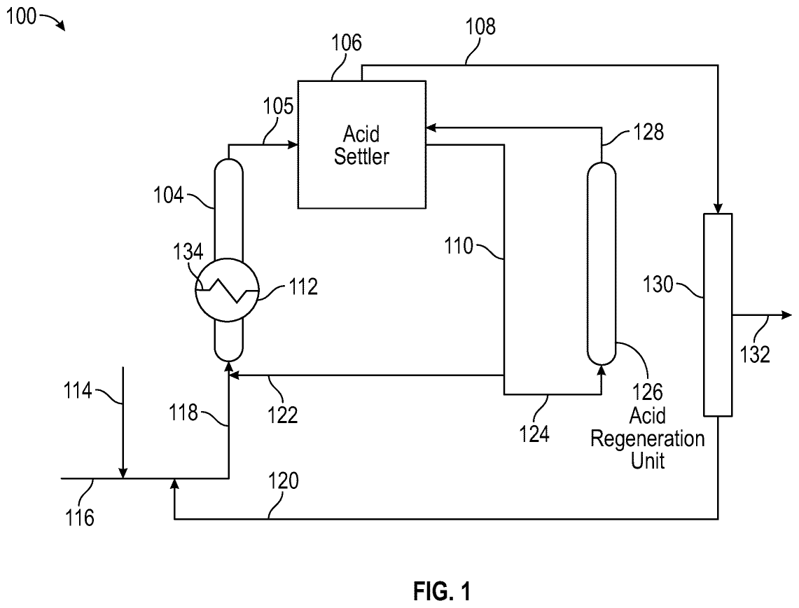

With reference to FIG. 1, alkylation may be performed in a reaction system 100 or process utilizing appropriate equipment. An olefin feed stream 114 is mixed with an isoalkane feed 116 to form an olefin/isoalkane mixture 118. A recycled isoalkane feed 120 may optionally be added to the olefin/alkyl mixture (or alternatively added to the olefin feed stream 114 or the isoalkane feed stream 116 before mixing). The olefin/isoalkane mixture stream 118 is then contacted with the acid feed stream 122. The acid feed stream preferably has a strength of about 80 wt. % or greater. The acid catalyzes the alkylation reaction of the olefin feed in the reaction vessel 104. During the reaction, the temperature of the material (i.e., feed and products) in the reaction vessel 104 increases, as the reactions are exothermic. Much of the heat of reaction is removed by externally cooling the acid prior to mixing with the olefin/isoalkane, or by direct heat transfer with cooling media within the reaction vessel 104 or associated cooler 112. The cooling load 134 of cooling the acid is represented as a heat exchanger, though it should be understood that this represents heat removal in general and should not be limited to only a heat exchanger. The product stream 105 including spent acid, excess isoalkane, byproducts of unfavored side reactions (not the primary alkylation reaction), contaminants/inert hydrocarbons, and produces alkylate exits the reaction vessel 104 and is provided to an acid settler 106. The alkylation reaction is exothermic. As a result, the product stream 105 has a higher temperature than the feed streams.

The acid settler 106 separates the acid and other material in the product stream 105. Within the acid settler 106, the product stream 105 is separated into other streams by one or more separation processes including, for example, distillation and/or gravimetric separation. Excess isoalkane reactant 108, such as iso-butane, is captured and can be purified in a stripper, fractionator, or other appropriate equipment 130, then recycled to the alkylation reaction as a recycled isoalkane stream 120. The stripper/fractionator 130 can also result in one or more product streams 132 containing the alkylate. Excess acid temperature can favor the formation of unwanted by products, such as fluorinated or sulfonated hydrocarbon molecules. Thus, spent acid 110 is collected in the acid settler 106 in a portion of the settler known as the acid boot. Some portion of the spent acid may also be removed from the system from the stripper 130 or any of the various acid-containing streams. The spent acid 110 may optionally pass through the acid cooler 112 that serves to lower the temperature of the acid. The spent acid may be partitioned into the acid feed stream 122 and a recycle portion 124.

The recycle portion of the spent acid 124 from the system is collected, bypasses the reaction vessel 104 and is sent to an acid regeneration unit 126 so the acid strength can be maintained by removing impurities that dilute the acid such as water and acid soluble oils (ASO). A stripping isoalkane stream is provided to the acid regeneration unit (not shown) that allows the acid to volatilize within the regeneration unit, leaving the impurities behind. Regenerated acid 128 can be recycled to the acid settler or to another part of the system for reuse as a catalyst.

It is important to maintain the correct acid strength level in the alkylation system. Low acid strength can result in the formation of undesirable byproducts, such as acid soluble oils (ASO) and excess water formation. To prevent the formation of ASO and other undesired byproducts, operators target a minimum acid strength of about 80 wt. % or higher. Acid strength may range from 80 wt. % or higher for it to be effective. In some processes, the acid strength is maintained at 80-90 wt. % or greater. In other processes, the acid strength is maintained at 85-90 wt. % or higher. Fresh acid is added to a process to restore the acid to the desired level of strength for reuse in the alkylation process, at an amount that compensates for loss of acid within the process, typically caused by formation of byproducts or intermediate products that result from the alkylation reaction not reaching completion, or lack of efficiency in the acid regeneration unit 126.

Weak acid conditions in the alkylation unit can lead to runaway reaction conditions. Weak acid conditions favor the formation of ASO and other byproducts such as water, which further weakens the acid. Weak acid catalyst conditions can also cause the buildup of reactive intermediates which can suddenly react, causing localized temperature spikes. Thus, it is important that the strength of the acid feed to the alkylation reactor be closely monitored.

However, the present methods and systems include the monitoring of other operating properties of an alkylation process to detect problem conditions. The signal provided by these other properties may be more subtle than the acid strength, but it has been found that monitoring other properties can also provide an operator with useful insight into the functioning of the alkylation process. Early detection of problems can allow and operator to prevent the formation of operating conditions that result in a runaway reaction, thereby preventing losses of feedstock and increasing economic efficiency of the alkylation process.

For example, the purity of the isoalkane recycle stream on a mass or molar basis can be indicative of potential runaway conditions, with lower purity indicative of excess hydrocarbons that are inert in the alkylation reaction being present in the reactor and therefore a greater risk of accelerated formation of ASO and dilution of acid strength, leading toward runaway conditions. Reliance on volumetric flow rate alone is not dependable, as byproducts may also be present in the stream. Other properties useful in detecting runaway conditions or in evaluating the risk of developing runaway conditions may include: the total isoalkane to olefin feed rate, the isoalkane feed rate, the acid to hydrocarbon feed ratio, fresh acid inventory available, sulfur in the feed streams to the alkylation unit, acid settler levels, reaction vessel temperature, downstream fractionator or separator acid vessel/boot valve status, alkylate boiling end point, alkylate residue after distillation, alkylate to barrel olefin feed ratio, and alkylate color. Monitoring such properties can assist an operator of an alkylation process in understanding the operation status of the process and can assist an operator in identifying when a process shows signs of potential runaway acid conditions within the process. By preventing or by intervening early on in the onset of runaway conditions, the process can be corrected thereby increasing the overall material and economic efficiency of the process.

The factor and associated methods described herein differ from conventional single-parameter alarms and typical distributed control system (DCS) configurations in several respects. Conventional systems often monitor individual variables against fixed alarm limits and respond only when a particular variable exceeds its respective limit. By contrast, the methods disclosed herein can combine a plurality of operating parameters from different parts of the alkylation system—including, for example, feed quality parameters, reactor conditions, acid regeneration status, and downstream product quality metrics—into a single composite factor. The composite factor can be configured to represent an overall operational health or risk level of the unit, even when individual parameters remain within their individual specification ranges. This cross-unit, composite evaluation of the operating state enables earlier and more holistic detection of abnormal operating conditions.

### Property Monitoring Method

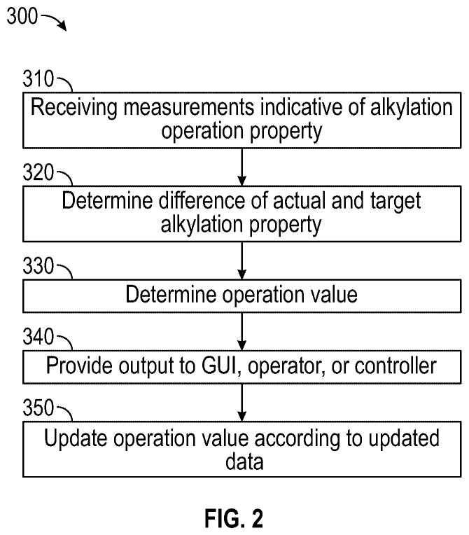

Turning now to FIG. 2, a method of monitoring an alkylation system is provided herein. At a high level, the method 300 includes monitoring operating properties of an alkylation system and assigning a factor to each property, depending on how that property falls within a designated operating range. Then, the factors assigned for each designated operating property are compiled into a single composite factor or composite value. This overall composite value is used to determine what steps should be taken to ensure continued efficient operation of the alkylation process. The method is performed at least in part by a control system associated with alkylation process, the control system including one or more processors and one or memory. Some steps may be performed by the control system or by an operator of the alkylation process. The method may include several steps performed sequentially or at the same time. Although represented in a sequential manner in FIG. 2, it should be understood that the steps of the method are not required to be performed sequentially. Steps may be performed a single time or may be performed multiple times as part of the method.

In general, the term “factor” as used herein refers to any value derived from one or more operating parameters of the alkylation system that is indicative of an operational status of the system. The factor may be a single scalar value (for example, an index ranging between a minimum and a maximum value), a multi-dimensional vector of values associated with different sub-aspects of the process, or a categorical indicator such as a qualitative risk level or health state (e.g., “normal,” “alert,” “critical”). In some embodiments, the factor is indicative of a likelihood of onset of runaway acid conditions in the alkylation system. In other embodiments, the factor is indicative of one or more of overall catalyst health, expected alkylate product quality, process efficiency, or a combination thereof. Unless expressly stated otherwise, the term “factor” also encompasses composite values determined according to two or more operating parameters.

In a general aspect, which can be combined with any other aspect or embodiment provided herein, the method 300 includes a step 310 receiving data representative of the alkylation process operating parameters (monitored properties). This data includes operation set points (e.g., target operating temperatures, flow rates, etc.) and actual or recent factors (e.g., live temperature, last known flow rate, etc.) of the monitored properties of the alkylation process. The data is received, by one or more processors and from one or more sensors, the data representative of measurements of two or more operating properties of an alkylation system. Data received includes data for one or more properties, and preferably for two or more properties. Data on more than two properties can be received (e.g., three properties, twenty properties, fifty properties, etc.). In some embodiments, data is received for several operating properties.

Data may additionally or alternatively be received from operator inputs and/or secondary sources. Such data may include data provided by experiments performed on ex situ samples. For example, acid strength may be determined by laboratory testing. In such a case, data regarding the acid strength may be provided via a user input to the control system or it may be stored in a secondary data source (i.e. in a system available to both the laboratory and the control system). Data may be transmitted via wireless or wired communication methods as known in the art.

The receiving data step may be performed periodically, and may have different periods of time for each property received. For example, temperature measured via a thermocouple may be updated very frequently such as every 10 seconds, every 5 second, every 1 second, or less. A laboratory-based acid strength measurement may be updated every 30 minutes, every hour, every 24 hours, or more.

The method 300 includes a step 320 of determining a difference between the measured values and target values for one or more of the monitored properties. The target values for each operating parameter or property are typically represented as a range. The differences are determined by the control system by the one or more processors. For this step, it is preferable that the difference between the measured and target values of at least two properties are determined. The differences are indicative of how far away from optimal operation the alkylation system is. The difference may be determined for each monitored property or for a group or subset of monitored properties, or for a single property. The determination can be repeated such that the difference can be updated when the underlying property data is updated, or can be updated independent of the property update frequency. For example, the method may include comparing or otherwise processing data related to the temperature of reaction zone of an alkylation process and an acid strength associated with the acid in an alkylation process. These data can be processed to generate a difference value for each property.

In some embodiments, the target values or target ranges for one or more operating parameters are not fixed but are adjusted automatically by the control system based on changes in operating conditions. For example, if the feed composition shifts towards a higher olefin content, the control system may adjust the target isoalkane-to-olefin ratio or acid:hydrocarbon ratio to maintain desired reaction selectivity. Similarly, if ambient temperature or cooling capacity changes, the control system may adjust the target reactor temperature range. The factor is then determined based on the updated target values, allowing the monitoring and control strategy to adapt to evolving conditions without requiring continuous manual reconfiguration.

The method 300 includes a step 330 of determining a factor associated with two or more of the one or more monitored properties, the factor being based on the difference of the two or more monitored properties and the corresponding target monitored properties, as determined in step 320. The determination of the factor is performed by the one or more processors. Determining a factor can assist an operator in identifying operating properties indicative of reduced alkylation reaction efficiency, buildup of ASO or other contaminants in the system, or other operational issues related to the alkylation system. In some embodiments, the factor is a value based on a single difference. This may be useful, for example, when the determined difference for a particular property is large enough that it controls which steps should be taken in other steps. As an example, if the flow rate of isoalkane reactant to the alkylation process is found to have fallen below a critical value, that fact may be determinative of which further steps are taken (e.g., reducing flow of olefin feed, or shutting down the process, etc.).

The factor can be determined in different ways depending on the implementation. In some embodiments, the factor is determined using deterministic rules, for example by assigning discrete scores or points to ranges of operating parameter deviations and summing the scores. In other embodiments, the factor is determined as a continuous function of the operating parameters, such as a weighted linear combination, a piecewise-linear function, or a non-linear function (for example, a quadratic or logarithmic expression) of one or more differences between measured values and their corresponding targets. In still other embodiments, the factor is determined at least in part using a data-driven or model-based approach, such as a regression model, a decision-tree model, a neural network, or another machine-learning algorithm trained to associate operating parameter data with a desired indication of process status or risk.

The type, function, nature, or interpretation of the factor may vary among implementations. In some embodiments, the factor represents a risk index that increases as operating conditions become more conducive to runaway acid reactions or formation of undesirable byproducts. In other embodiments, the factor represents a catalyst health index that provides an indication of the degree of contamination, deactivation, or dilution of the acid catalyst. In yet other embodiments, the factor represents a performance index that correlates with one or more economic or quality-related measures, such as alkylate yield per unit mass of acid consumed, expected octane number of the alkylate product, or expected off-spec production rate. In certain implementations, different factors or indices are determined in parallel, each reflecting a different aspect of the alkylation process, as described further herein.

In some embodiments, the factor is a composite value according to two or more of the determined differences of step 320. The method may include determining any desired number of composite values as may be of assistance to an operator. In some embodiments, the control system is configured to determine multiple factors associated with different aspects of the alkylation process. As non-limiting examples, the system may determine an “acid health index” indicative of acid strength and contamination, a “reaction severity index” indicative of reaction vessel temperature and isoalkane-to-olefin ratio, a “product quality index” indicative of alkylate boiling end point, residue, and color, and a “unit reliability index” indicative of equipment conditions such as acid settler levels and downstream acid boot valve position, or other indices as desired by an operator. Each of these indices may itself be a composite value determined according to two or more operating parameters. The system may then determine an overall factor based on a combination of these indices, or may present the indices separately to an operator to provide more granular insight into which aspects of the alkylation system are contributing to an elevated overall risk level.

In some embodiments, the method includes determining a single composite value. In other embodiments, the method includes determining multiple composite values, each determined according to a different set of measured properties and/or user inputs. In determining a composite value, some properties may be weighted differently than others, providing higher weight to properties that are more important to the operation of the alkylation system or those that have differences that merit higher weight. Indeed, the weight that a property is given in the composite value may be variable as a function of the underlying differences, or any other desired variable (for example, time, production rates, exterior temperatures, etc.). In some embodiments, the factor also incorporates dynamic weighting and temporal trend information that are not typically used in standard alarm systems. For instance, the contribution of a given operating parameter to the factor may increase if the parameter deviates further from its target range, if the deviation is changing rapidly, or if the deviation has persisted for longer than a selected time interval. Additionally, the relative weight assigned to different parameters in determining the factor may be adjusted based on process context. As one example, when acid strength falls below a critical value, the control system may assign a higher weight to acid strength and related parameters so that the factor becomes primarily sensitive to acid-related risk, while temporarily reducing the contribution of less critical parameters. This context-dependent, trend-sensitive computation allows the factor to reflect both the magnitude and the urgency of process deviations in a way that is not achieved by static, single-variable alarm limits.

In some embodiments, the operating parameters used to determine the factor include a rate of change of at least one process variable. For example, the control system may calculate a temperature ramp rate for the alkylation reaction vessel, such as degrees Fahrenheit per minute, based on successive temperature measurements. The factor may increase more rapidly when the temperature ramp rate exceeds a predetermined threshold, even if the absolute temperature remains within its target range, thereby providing an indication of rapidly intensifying reaction conditions. Similarly, a rate of change in sulfur content, acid strength, or isoalkane recycle purity may be included as an input to the factor to detect rapid deterioration in feed or catalyst quality.

In certain implementations, the factor includes a time-weighted persistence component associated with one or more operating parameters. For instance, for each parameter that is outside its target range, the control system may accumulate a persistence score that increases as a function of the duration of the deviation. The contribution of that parameter to the factor may then be based on both the magnitude of the deviation and the accumulated persistence score. This approach allows the factor to reflect the cumulative effect of prolonged small deviations as well as short-lived large deviations, and can be used to prioritize intervention when a parameter has been unfavorable for an extended period even if its current deviation is modest.

In some embodiments, different operating parameters are assigned different weighting coefficients depending on their relative importance to the overall health or risk state of the alkylation system. For instance, parameters directly associated with acid strength and reaction temperature may receive higher weights than parameters associated with secondary quality indicators. The weighting coefficients may be fixed, may be defined by an operator, or may be adjusted dynamically by the control system, or a combination of any two or more thereof. In certain implementations, the weighting coefficients are context-dependent; for example, when acid strength falls below a first threshold, the control system may increase the weight associated with acid strength and related parameters so that the factor becomes more sensitive to further acid degradation, while reducing the weights of less critical parameters. Similarly, when a sulfur spike in the feed is detected, the system may temporarily increase the weight of total sulfur and alkylate residue so that the factor reflects increased risk of undesirable byproduct formation.

In some embodiments, the factor is computed using a continuous function of one or more differences between measured operating parameters and their corresponding target values. For example, a factor (F) may be determined according to:

$$F = {\sum }_{i} {w}_{i} \cdot {f}_{i} ( Δ {P}_{i} )$$

where ΔPᵢ is a difference between a measured value and a target value for operating parameter i, wᵢ is a weighting coefficient associated with operating parameter i, and ƒᵢ is a mapping function that converts the difference into a contribution to the factor. The mapping function ƒᵢ may be linear (e.g., proportional to ΔPᵢ), piecewise-linear (e.g., using different slopes in different deviation ranges), non-linear (e.g., quadratic, exponential, logarithmic), or stepwise (e.g., assigning discrete scores to different bands of deviation). In some embodiments, the factor is normalized or scaled to a predetermined range, such as 0 to 100, to facilitate interpretation by an operator.

The method 300 includes a step 340 of taking action according to the determined factor. This step may include, for example, but is not limited to in response to the factor: displaying a value on an operator interface, activating an audio or visual alarm, sending a signal to a piece of equipment associated with the alkylation process, sending a notification to an operator, calling a phone number, sending an email message, updating a database on a storage medium, changing the operating parameters of a valve or other equipment of the associated alkylation system (e.g., opening a valve some amount, or slowing a pump, stopping a flow of a stream, etc.), changing a flow rate of a reactant to the alkylation system, changing a flow rate of acid catalyst to the alkylation system, or changing an operating temperature associated with the alkylation system. The taken action may include stopping a flow of olefin and/or stopping a flow of acid feed to the system.

In some embodiments, the factor is used not only to report a current state of the alkylation system but also to drive preventive control actions. For example, the control system may be configured such that a first range of factor values corresponds to normal operation, a second range corresponds to a cautionary state in which one or more operating parameters are automatically adjusted to steer the process back toward the target ranges, and a third range corresponds to a critical state in which more aggressive corrective actions, including automated reduction of olefin feed or shutdown of acid feed, are executed. The use of the factor in this manner allows for graded, preventive interventions that can reduce the occurrence of runaway conditions, minimize production of undesired byproducts, and improve overall economic efficiency of the alkylation process.

In some embodiments, the method 300 includes determining the factor according to two or more properties and/or their associated differences. In some embodiments, the method 300 determines the factor according to a combination of two or more of the acid strength, the ratio of the total isoalkane to olefin feed rates, the isoalkane recycle feed rate, the acid to hydrocarbon feed ratio, fresh acid inventory available, sulfur in the feed streams to the alkylation unit, acid settler levels, reaction vessel temperature, downstream fractionator or separator acid boot valve status, alkylate boiling end point, alkylate residue after distillation, alkylate to barrel olefin feed ratio, alkylate color, or any two or more thereof. Thus, the method includes, in some embodiments, taking an action based on a composite value that is determined according to two or more of the properties listed above.

In some embodiments, the factor is determined according to only a subset of the operating parameters described herein. Any one or more of the operating parameters may be used alone or in combination with any other operating parameter to determine the factor. For example, in certain implementations the factor is based only on acid purity and alkylation reaction temperature. In other implementations, the factor is based only on downstream product quality parameters, such as alkylate boiling end point, alkylate residue, and alkylate color. In still other implementations, the factor is based only on one or more ratio-type parameters, such as the ratio of a feed rate of isobutane to a feed rate of olefin, the ratio of a feed rate of acid catalyst to a feed rate of hydrocarbon, or the ratio of a flow rate of alkylate to a feed rate of olefin. Accordingly, the particular set of operating parameters used to determine the factor can be selected based on the configuration of the alkylation unit, available instrumentation, and operator preference. In other embodiments, combinations of parameters are considered.

In some embodiments, the factor is used in conjunction with an economic objective function to determine control actions that both reduce risk and improve economic performance. For instance, the control system may receive or determine an estimate of alkylate value, acid consumption cost, and energy cost, and may evaluate candidate changes to feed rates, acid addition, or operating temperature with respect to both the resulting factor and the economic objective. Control actions may then be selected that maintain the factor below a predetermined limit while optimizing one or more economic metrics, such as maximizing alkylate yield per unit acid consumption or maximizing profit per unit of feed processed.

The method 300 may optionally include a step 350 of changing the factor according to updated data. In some embodiments, the factor is updated based on new or updated data received by the control system or user input. In some embodiments, the factor is updated on a fixed or variable time schedule. In some embodiments, the factor is updated in response to new data received by the control system. In some embodiments, the factor is changed in response to a change in the time of day, as in a scheduled event. In some embodiments, the factor is changed in response to new target values received by the control system for one or more operating parameters.

### Property Monitoring System

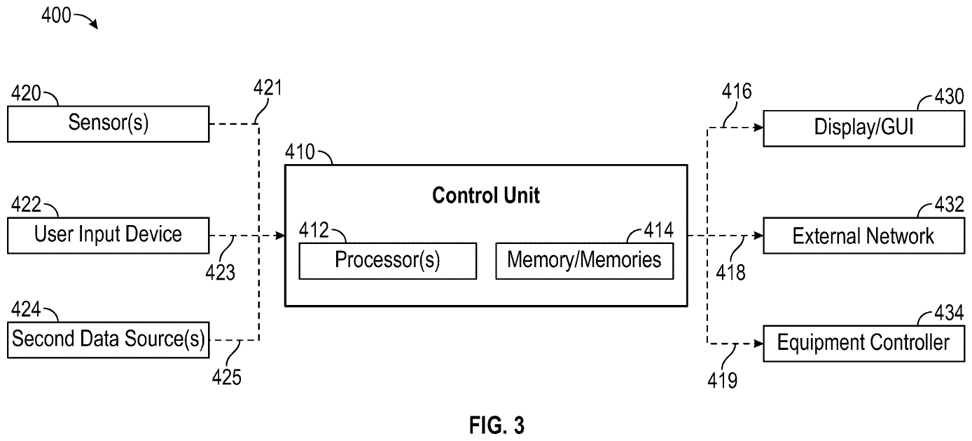

As described above, a method of monitoring an alkylation process may be carried out using one or more control systems associated with the alkylation process. Turning now to FIG. 3, disclosed herein is a system 400 for monitoring the operating properties of the alkylation process. In some embodiments, the system executes, at least in part, the method previously described. Each of the monitored operating properties may be measured and/or monitored during the alkylation process, and deviations from normal operating ranges can be detected and recorded. The monitoring system 400 includes one or more processors 412 and one or more computer memory 414. In some embodiments, the monitoring system 400 includes a control unit 410 or control circuit that includes the one or more processors 412 and the computer memory 414 structured to store instructions that, when executed by the one or more processors, cause the control circuit to: receive, by one or more processors and from one or more sensors 420 and/or from one or more operator input devices 422 and/or one or more secondary data sources 424, measurements indicative of one or more of the alkylation system operating properties; determine, by the one or more processors, one or more composite values according to the received measurements; and perform an action according to the determined composite value(s).

In some embodiments, one or more operator inputs are used as additional inputs to the factor. For example, an operator may manually input laboratory test results, observations of equipment status, or qualitative assessments of process performance through the user input device 422. Such operator inputs may be treated as operating parameters in the same manner as sensor-derived parameters and may be included in the computation of the factor. In other embodiments, operator inputs may be used to modify target ranges, weighting coefficients, or thresholds associated with particular parameters, thereby allowing the factor to reflect operator knowledge of current or anticipated conditions, such as planned maintenance activities, temporary changes in feed quality, or intentional changes in operating parameters or objectives.

In some embodiments, the factor is based in part on estimated values of one or more operating parameters that are not directly measured. For example, a process model, such as a mass and energy balance model, a thermodynamic model, or a kinetic model of the alkylation reaction, may be used to estimate an internal variable such as localized acid strength in the reactor, residence time distribution, or concentration of reactive intermediates. These estimated parameters may then be treated as additional operating parameters and used in the factor computation. In this way, the factor can capture aspects of the process state that are not directly observable from available sensors.

The property monitoring system 400 is configured to receive data 421 423 425 from one or more sensors 420 or from one or more operator inputs devices 422 or from one or more secondary data sources 424. In some embodiments, the sensors directly provide data 421 to the control unit 410 of the monitoring system 400. In other embodiments, the sensors 420 are connected to the control unit 410 through intervening control units/systems. The sensor data 421 may be transmitted to the control unit 410 via wired or wireless connections.

A user input device 422 may also provide data 423 to the control unit 410. The user input device 422 should not be limited to any particular arrangement—it may be a keyboard, mouse, touchscreen, voice recognizer, etc., or any device that allows a user or operator to provide data to a control system via input. In some embodiments, the user input device 422 is part of an operator workstation or is part of a laboratory workstation.

In some embodiments, the monitoring system 400 has access to or receives data 425 from secondary data sources 424. The secondary data source 424 may be a database, external memory, external storage, remote memory, remote storage, a value found online or on a website, etc. In some embodiments, the secondary data source 424 is a database that can be queried for data 425 relating to the operation of the alkylation system.

The data 421 423 425 provided by the sensors 420, operator input devices 422, and/or secondary data sources 424 can include data on operating parameters or properties of an alkylation system. These properties include, but are not limited to: the acid strength, the ratio of the total alkyl to olefin feed rates, the total isoalkane to olefin feed rate, the alkane recycle feed rate, the acid to hydrocarbon feed ratio, fresh acid inventory available, sulfur in the feed streams to the alkylation unit, acid settler levels, reaction vessel temperature, downstream fractionator or separator acid boot valve status, alkylate boiling end point, alkylate residue after distillation, alkylate to barrel olefin feed ratio, alkylate color, or any two or more thereof.

In some embodiments, the control system is configured to validate incoming measurements and to handle sensor faults or data quality issues when computing the factor. For example, the control system may compare values from redundant or closely situated sensors, apply plausibility checks based on mass or energy balances, or verify that parameter changes fall within realistic physical limits over a given time step. If a measurement is determined to be invalid or unavailable, the control system may exclude that measurement from the factor computation, substitute a value from a redundant or another closely situated sensor, or use an estimated value based on a process model or historical data. This fault-tolerant approach allows the factor to remain meaningful even in the presence of sensor failures or communication disruptions.

The acid strength is measured by conducting laboratory tests on samples collected from the alkylation unit or by using sensors located at one or more acid streams. For example, the acid streams may be those feeding or recycling acid within the alkylation system, such as 110 122 124. Such measurements could be performed by pH sensors, conductivity sensors, optical sensors, density sensors, or another sensor capable of providing data from which acid strength of the stream can be determined. In some embodiments, the purity of the isoalkane recycle stream is measured by conducting laboratory tests on collected samples or by using sensors 420 in one or more recycled isoalkane streams 108 120. Such measurements could be performed by gas chromatography, mass spectroscopy, optical sensors, or another sensor capable of providing data from which isoalkane concentration of the stream can be determined. Data 423 425 from these measurements could be made available to the system to receive via a user input device 422 or a secondary data source 424. In some embodiments, the acid strength data is provided by a combination of sensors, laboratory tests, user inputs, and/or secondary data sources.

The ratio of the total isoalkane to olefin feed rate can be determined by monitoring the feed flow rates of the olefin feed stream 114 and the isoalkane feed stream 116, for example. These data can be measured by any known volumetric and/or mass flow meters/sensors. The isoalkane recycle feed rate can be determined by monitoring the feed flow rate of isoalkane recycle stream 120, for example. These data can be measured by any known volumetric and/or mass flow meters/sensors or other suitable devices. The acid to hydrocarbon feed ratio can be determined by monitoring the feed flow rates of the combined feed stream 118 and the acid feed stream 122, for example. Or the acid feed rate can be calculated using temperature sensors in combination with known heats of reaction of the olefin feed components. These data can be measured by any known volumetric and/or mass flow meters/sensors or other suitable devices. The fresh acid inventory available is a measurement of the amount of fresh acid supply available to supplement the system as make up acid or to increase the strength of the acid in the system and, as such, can be measured by mass or volume available. It may also be useful to consider this parameter from the perspective of a ratio or percentage against the amount of acid in the alkylation system exclusive or inclusive of the fresh acid inventory (e.g., fresh acid available may be equal to 40% of the acid in active use in the alkylation system on a mass basis).

The amount of sulfur in the feed streams to the alkylation unit can be measured by laboratory tests such as combustion and/or UV analysis, gas chromatography, and/or mass spectroscopy; however suitable sensors may also be available. The acid settler levels can be measured by any sensor suitable for measuring liquid levels, such as floats, radar, sonic sensors, nuclear devices, etc. Data from such sensors can be used to determine the height of the acid levels in various portions of the acid settler. The reaction vessel temperature is a measurement of the temperature within the reaction vessel where the alkylation reactions mostly occur. As such, the reaction vessel temperature can be determined by appropriate thermocouples or other temperature sensors, or infrared imaging, either internal or external to the vessel.

The downstream fractionator/separator acid boot valve status can provide an indication of how open or closed the acid boot level control valve position is, which indicates how much spent acid 110 is exiting the settler 106. Higher values can be indicative of increased acid carryover into the fractionator/stripper. This property can be measured by any appropriate sensor, flow measurement device, or may be directly obtained by querying the valve in the of an electronically controlled valve. The alkylate boiling end point is a measurement of the final or end boiling point for a sample of the alkylate product, indicative of the size of hydrocarbon molecules contained therein. Higher boiling end points are indicative of higher amounts of ASO formation in the system. Such data can best be obtained by laboratory testing of samples obtained from the alkylation system, such as subjecting a sample of the alkylate product stream to distillation. Similarly, the alkylate residue after distillation is provided by laboratory testing of a collected sample of the alkylate. The sample is subjected to distillation and the amount of residue remaining after the alkylate has been volatilized is measured. The residue is primarily composed of long chain hydrocarbons, polymers, or other contaminants, and collecting this information provides useful insight into the performance of the alkylation system.

The alkylate to barrel olefin feed ratio can be determined by measuring the flow rate of alkylate product and comparing it to the amount of olefin feed provided to the alkylation system. The color of the alkylate can be determined by a sensor or by collecting a sample of the alkylate and performing lab testing thereon.

The monitoring system 400 is configured to determine, by the one or more processors 412, a difference between the property value provided by the data 421 423 425 and a predetermined target value. For example, the system may be configured to determine the difference between the reported alkylation reaction vessel temperature and a target alkylation reaction vessel temperature. The difference may be static (i.e., independent of time or only according to the most recent value) or may be time-dependent (i.e., may be determined according to the most recent and also some or all past data points). As such, the difference may represent a change over time in a property value. Time dependent differences may be made according to data that has aged up to 1 second, up to 5 seconds, up to 10 seconds, up to 30 seconds, up to 1 minute, up to 2 minutes, up to 5 minutes, up to 10 minutes, up to 15 minutes, up to 20 minutes, up to 30 minutes, up to an hour, up to 2 hours, up to 3 hours, up to 4 hours, up to 5 hours, up to 6 hours, up to 8 hours, up to 10 hours, up to 12 hours, up to 24 hours, up to 48 hours, or longer.

The factor may further incorporate temporal information related to the operating parameters. In some embodiments, the operating parameters include not only instantaneous values but also time-dependent values such as rates of change or moving averages. For example, the control system may determine a rate of change of the alkylation reaction temperature (e.g., ° F. per minute/5 minutes/10 minutes/etc.) or a rate of change in acid strength over time, and may include these rates as additional inputs to the factor computation. In other embodiments, the factor includes a persistence component that increases when an operating parameter remains outside its target range for more than a selected duration. Thus, a brief excursion beyond a target range may contribute less to the factor than a deviation that persists for several minutes or hours. Time-dependent contributions may be determined using time windows ranging from seconds to hours, depending on the dynamics of the monitored parameter.

The monitoring system 400 is configured to determine, by the one or more processors 412, one or more factors according to some or each of the measured properties. In some embodiments, the monitoring system 400 is configured to determine a factor according to one or more of the measured properties and/or one or more user inputs and/or secondary data values. The monitoring system 400 may be configured to determine any desired number of factors, as will be of assistance to an operator. In some embodiments, the monitoring system 400 is configured to determine a single factor. In other embodiments, the monitoring system 400 is configured to determine a composite value, which is a factor based on the differences determine for more than one property. In some embodiments, the monitoring system 400 is configured to determine more than one composite value, each determined according to a different set of measured properties and/or user inputs and their associated differences.

The monitoring system 400 is configured to determine a composite value and may be configured to weigh some properties differently than others, providing greater weight to properties that are more important to the operation of the alkylation system or whose values/differences may indicate a higher priority issue in the alkylation system. In some embodiments then, the composite value is determined according to two or more properties but is driven primarily by the value and/or difference in a value that is determined to be the most important at a given time. In other words, the system may determine a factor that is a composite value but that is largely or completely driven by one or more values that indicate a priority issue that must be addressed in the alkylation system. For example, if the acid strength drops below a critical value, the monitoring system 400 may largely ignore the values, changes, and differences associated with other monitored properties and may give such weight to the acid strength such that the composite value is largely determined by the acid strength value/difference. Determining a composite value can assist an operator in identifying operating properties indicative of reduced alkylation reaction efficiency, buildup of ASO or other contaminants in the system, or other operational issues related to the alkylation system.

When determining the factor(s), the system may be configured to weigh the value and the amount of deviation from the normal operating ranges or target value, i.e., to give greater weight to values/differences that are more important or that indicate an urgent issue in the alkylation system. Thus, the factor can be used to indicate an appropriate action to take in response to a value, change, or difference of a property. For example, the system may determine a factor according to how far below the measured acid strength is from 90 wt. % and the amount of reserve acid available for the system, and may weigh the acid strength more strongly than the amount of reserve acid.

In some embodiments, the control system stores historical values of the operating parameters, the factor, and any control actions taken in a data store or database. The stored data may be used for later analysis, for example to identify recurring patterns that preceded past instances of runaway conditions or off-spec product. In certain implementations, the stored data is used to develop or update predictive models, such as regression models, decision-tree models, or neural-network models, that estimate future values of the factor or directly estimate a probability of undesirable events based on current and recent operating conditions. The control system may apply such a predictive model in real time to anticipate an increase in the factor and to implement preventive adjustments to the alkylation process before critical thresholds are reached.

In some embodiments, the monitoring system 400 is configured to perform an additional step in response to one or more factors reaching predetermined values. The factors, as discussed, may be composite values that are determined according to two or more properties. For example, in some embodiments, the monitoring system 400 is configured to determine a factor according to data associated with the acid strength, and the determined factor for an acid strength falling below 80 wt. % triggers the monitoring system 400 to take an action, such as sounding an audio alarm to alert an operator. In some embodiments, the monitoring system 400 is configured to send a signal 416 to a display or graphical user interface (GUI) 430 in response to one or more of the factors. For example, the monitoring system 400 may display a warning on an operator screen to notify the operator of less than ideal conditions in the alkylation system that may negatively impact productivity of the alkylation process.

The graphical user interface 430 may provide additional visualization and guidance related to the factor. For example, the GUI may display a time-series trend of the factor and individual operating parameters, a bar chart illustrating the contribution of each parameter to the current factor, and color-coded indicators to distinguish normal, caution, and critical ranges. In some embodiments, the GUI also displays recommended actions associated with the current factor value or trend, such as suggested adjustments to acid strength, isoalkane feed rate, reactor temperature, or other operating conditions. In certain implementations, the system includes a simulation or “what-if” mode in which an operator can input hypothetical changes to one or more operating parameters or setpoints and view the predicted impact on the factor without affecting the live alkylation process.

In some embodiments, the monitoring system 400 is configured to send a signal 418 to an external network 432 in response to one or more of the factors. For example, the monitoring system 400 may be configured to send a call to a cellular phone associated with an operator of the alkylation system in response to a determined factor. In some embodiments, the monitoring system 400 is configured to send a signal 419 to one or more pieces of equipment or equipment controllers associated with the alkylation system in response to one or more of the factors. For example, the monitoring system 400 may be configured to send a signal to a valve or a valve controller, instructing the valve to adjust to a determined open value, thereby affecting the flow rate of a feed stream in the alkylation system.

In some embodiments, the monitoring system 400 is integrated with one or more higher-level plant control or information systems. The factor may be used by such systems to prioritize maintenance activities, to schedule acid regeneration campaigns, or to coordinate operation of multiple alkylation units. In other embodiments, the factor and selected operating parameters are transmitted over a network, such as a local area network or a secure wide area network, to a remote monitoring center, where operators and/or subject matter experts can review real-time and historical data for multiple units and provide guidance or override commands back to the local control system.

In refineries having multiple alkylation trains or units, the factor may be used to coordinate operation among the units. For example, the monitoring system may compute a separate factor for each unit and may compare the factors to identify which unit is operating under more stressed or higher-risk conditions. The control system may then adjust feed distribution among the units, such as diverting a portion of olefin feed from a unit having a higher factor to a unit having a lower factor, in order to balance risk and maintain overall production. In some embodiments, the factors from multiple units are combined into a plant-wide index that assists operators in managing refinery-wide alkylation performance.

In some embodiments, the system is configured to perform a first step in response to a first measured property reaching or exceeding a first predetermined threshold value and to take a second additional step in response to a second measured property reaching or exceeding a second predetermined threshold value. In such embodiments, the first and second property may be the same property, or they may be different properties. The use of a first and second property and first and second additional steps is merely for convenient illustration—the system may be configured to take any number of additional steps based on any number of properties reaching or exceeding predetermined values (i.e., a third, fourth, fifth step, etc. in response to a third, fourth, fifth property value, etc. reaching a corresponding predetermined value). The additional steps can be sequential or may be taken at or about the same time. The additional steps may be dependent on previous steps having been performed, or may be taken independently of other steps.

In some embodiments, the system is configured to perform an additional step in response to a determined factor reaching or exceeding a predetermined threshold value. For example, in some embodiments, the system is configured to determine a composite value factor according to the acid strength, the reaction vessel temperature, and an operator input and to provide an output signal in response to the composite value exceeding a predetermined value. The predetermined value can be provided by the system (i.e. stored in memory 414), received from another system or secondary data source 424, or provided by an operator input via an user input device 422 (keyboard, mouse, etc.). In some embodiments, the monitoring system 400 is configured to perform a first step in response to a first factor as determined by the monitoring system reaching or exceeding a first predetermined threshold value and to take a second additional step in response to a second determined factor reaching or exceeding a second predetermined threshold value. In such embodiments, the first and second factors may be composite values, and may be the same composite value, or they may be different composite values. The use of a first and second factor and first and second additional steps is merely for convenient illustration—the system may be configured to take any number of additional steps based on any number of factors/composite values reaching or exceeding predetermined values (i.e., a third, fourth, fifth step, etc. in response to a third, fourth, fifth property value, etc. reaching a corresponding predetermined value). Additionally, the use of “reaching” or “exceeding” is for illustrative purposes only—the steps may likewise be taken in response to a factor falling below or simply being about equal to a predetermined value. The step may be taken in response to a factor maintaining a certain value for a predetermined period of time (e.g., the temperature of the reaction vessel exceeds predetermined temperature limit for more than 2 minutes, etc.). The additional steps can be sequential or may be taken at or about the same time. The additional steps may be dependent on previous steps having been performed, or may be taken independently of other steps.

### EXAMPLES

Examples are provided below. Although specific numerical target values and ranges are provided in one or more examples herein (for instance, target acid strength values, target isoalkane-to-olefin ratios, target temperature limits, or target residue levels), such values are intended to be illustrative and may be varied depending on the particular unit design, catalyst type, and desired product specification. For example, in some embodiments, the target acid strength may be between about 80 wt. % and about 90 wt. % or between about 85 wt. % and about 95 wt. %, and different bands within this range may be associated with different levels of contribution to the factor. Similarly, different target ranges and bands may be established for the isoalkane-to-olefin feed ratio, reaction temperature, or other monitored parameters, and these bands may be used to define multiple thresholds corresponding to low, medium, and high levels of risk or deviation. The factor can be configured to respond differently to each band, providing graded sensitivity to different degrees of deviation from the targets.

### Example 1—Method of Monitoring an Alkylation System

An alkylation process was modified to implement a method of monitoring the alkylation process. The alkylation process was performed at a refinery located in the United States. The process fed isobutane (iC4) as the alkylation reactant and the olefin feed was a mixture of olefins primarily comprising isobutylene and propene. Hydrofluoric acid (HF) was used as the catalyst. The purity of the HF feed to the reaction vessel and the purity of the iC4 recycle were sampled every hour and determined by laboratory testing. The data from these tests were provided to the system via user input. Samples of the alkylate product were routinely tested and the boiling end point, distillation residue, and alkylate color were recorded and utilized in the monitoring method.

In addition, data provided by sensors in the alkylation processed was utilized. This data included the amount of fresh acid in inventory, the temperature of the alkylation reaction vessel, the level of acid in the acid settler, and the downstream fractionator/separator acid boot valve percent open. Other sensor data was used to determine the ratio of the alkylate yield per barrel of olefin feed, ratio of acid to hydrocarbon ratio (acid:HC ratio) in the reaction vessel on a volumetric basis, the total amount of sulfur in the feed, and the alkylate yield per barrel of feed (alkylate:BB yield). Total iC4:olefin feed ratio (iC4:O ratio) on a volumetric basis was determined by measuring the volumetric flow rates of the olefin feed, its olefin content, and the volumetric flow rate of the combined (fresh and recycle) iC4 feed with its isobutane content. The acid regenerator unit (acid regeneration unit) status, whether in operation or not, was also monitored and taken into account.

For each of the above process properties, a target value or target range was assigned. The acid purity was assigned a target value of 87 wt. %, iC4 recycle purity a target value of 80 vol. %, total I:O a target value of 6.5 vol/vol, acid regenerator unit status a target value of on or operational, the acid:HC ratio a target value of 4 vol/vol, the fresh acid inventory a target value of 35 vol. %, the settler acid level a target value of 45-55% of maximum, the total feed sulfur a target value of less than 20 ppm by weight, the reaction vessel temperature a target value of less than 110° F., the alkylate:BB yield to a target range of 1.9 to 2.0, a downstream fractionator acid boot valve open percentage range of 30-35% open, an alkylate boiling end point at a target value of less than 400° F., the alkylate residue at a target range of 1.0-1.2 vol. %, and the alkylate color at a target value of greater than 30 Saybolt.

The properties were monitored and a factor was determined periodically. The factor was a composite value, determined according to all of the monitored properties. In this example, the composite value was called an Acid Runaway Index (ARI), with higher ARI values indicating issues in the alkylation process that could require attention. The ARI was a composite value determined by determining the difference between the most current data point for each property and the target value or range for that property. Greater deviations from the target value or range resulted in a larger contribution to the ARI value. In this example, the deviations of each monitored property were classified into one or four tiers, depending on the severity of the deviation. For example, the method would determine that the acid strength would contribute 1 point to the ARI when the acid strength fell within a value of 86-87 wt. % and would contribute 9 points to the ARI when the acid strength fell within a range of 80-84 wt. %.

According to the method as practiced, when the ARI reached a value of 10, the method proceeded to provide an alert to the operator that recommended reducing the feed of olefin to the alkylation system or that recommended increasing the acid strength. And when the ARI reached a value of 20, the alert would be provided to the operator that recommend shutting off the olefin feed to prevent runaway conditions from forming in the process.

### Example 2—System for Monitoring an Alkylation System

The method of Example 1 was carried out using a modified alkylation process system. The system included hardware including a control unit having computer processors, memory, and storage. The system also included a user interface in the form of a keyboard and mouse, and a GUI that displayed data to an operator and permitted the operator to change various parameters associated with the system. The system included communication devices that permitted the control unit to send and receive signals from equipment controllers, particularly those associated with various process valves and pumps. Thus, an operator could control the open percentage of a process valve or toggle the operation of a pump from the GUI. In this way, an operator could shut off feed of acid or olefin to the alkylation reactor. Other aspects of the alkylation process, such as supply of acid, flow rates of recycle streams, and flow rates of cooling water provided to the process could also be controlled by the operator via the GUI.

The GUI also included a display that updated to display to the operator a summary of the ARI and a table that illustrated the contribution of each monitored property to the ARI. The system included software that would display a recommendation or a warning to the operator when the ARI reached a predetermined value. For example, the system was configured to recommend add additional HF acid to the system from the fresh acid inventory or to increase the flow rate of iC4 to the acid regenerator unit to further strengthen the acid when the ARI reached a value of 5. The system was further configured to reduce or shut off the olefin feed to the alkylation process when the ARI reached a value greater than 20. The system was also configured to take one or more of the remediation steps automatically in response to the ARI value reaching 20.

While certain embodiments have been illustrated and described, it should be understood that changes and modifications can be made therein in accordance with ordinary skill in the art without departing from the technology in its broader aspects as defined in the following claims. All examples provided above, either indicated as examples or through the use of “e.g.” are for illustrative purposes and for clarity of the disclosure. These examples should not be considered to be limiting the disclosure in any way.

The embodiments, illustratively described herein may suitably be practiced in the absence of any element or elements, limitation or limitations, not specifically disclosed herein. Thus, for example, the terms “comprising,” “including,” “containing,” etc. shall be read expansively and without limitation. Additionally, the terms and expressions employed herein have been used as terms of description and not of limitation, and there is no intention in the use of such terms and expressions of excluding any equivalents of the features shown and described or portions thereof, but it is recognized that various modifications are possible within the scope of the claimed technology. Additionally, the phrase “consisting essentially of” will be understood to include those elements specifically recited and those additional elements that do not materially affect the basic and novel characteristics of the claimed technology. The phrase “consisting of” excludes any element not specified.

The present disclosure is not to be limited in terms of the particular embodiments described in this application. Many modifications and variations can be made without departing from its spirit and scope, as will be apparent to those skilled in the art. Functionally equivalent methods and compositions within the scope of the disclosure, in addition to those enumerated herein, will be apparent to those skilled in the art from the foregoing descriptions. Such modifications and variations are intended to fall within the scope of the appended claims. The present disclosure is to be limited only by the terms of the appended claims, along with the full scope of equivalents to which such claims are entitled. It is to be understood that this disclosure is not limited to particular methods, reagents, compounds, or compositions, which can of course vary. It is also to be understood that the terminology used herein is for the purpose of describing particular embodiments only, and is not intended to be limiting.

In addition, where features or aspects of the disclosure are described in terms of Markush groups, those skilled in the art will recognize that the disclosure is also thereby described in terms of any individual member or subgroup of members of the Markush group.

As will be understood by one skilled in the art, for any and all purposes, particularly in terms of providing a written description, all ranges disclosed herein also encompass any and all possible subranges and combinations of subranges thereof. Any listed range can be easily recognized as sufficiently describing and enabling the same range being broken down into at least equal halves, thirds, quarters, fifths, tenths, etc. As a non-limiting example, each range discussed herein can be readily broken down into a lower third, middle third and upper third, etc. As will also be understood by one skilled in the art all language such as “up to,” “at least,” “greater than,” “less than,” and the like, include the number recited and refer to ranges which can be subsequently broken down into subranges as discussed above. Finally, as will be understood by one skilled in the art, a range includes each individual member.

All publications, patent applications, issued patents, and other documents referred to in this specification are herein incorporated by reference as if each individual publication, patent application, issued patent, or other document was specifically and individually indicated to be incorporated by reference in its entirety. Definitions that are contained in text incorporated by reference are excluded to the extent that they contradict definitions in this disclosure.
A. A method comprising: receiving, by one or more processors and from one or more sensors, measurements indicative of one or more operating parameters of an alkylation process; determining, by the one or more processors, a difference between the one or more operating parameters of the alkylation process and one or more target operating parameters of the alkylation process; determining, by the one or more processors, a factor associated with one or more of the one or more operating parameters, wherein the factor is based on the difference; and providing, by the one or more processors, an indication of the factor to an operator of the alkylation process.
B. The method of Paragraph A, wherein the factor is a composite value determined according to two or more operating parameters.
C. The method of either one of Paragraphs A or B, further comprising changing a position of a valve, a flow rate of isobutane, a flow rate of catalyst, a flow rate of olefin, or an operating temperature associated with the alkylation process in response to the factor.
D. The method of any one of Paragraphs A-C, wherein the one or more operating parameters of the alkylation process include at least one of: an acid purity; an isobutane recycle purity; a ratio of a feed rate of isobutane to a feed rate of olefin; a feed rate of isobutane to a catalyst regenerator; a ratio of a feed rate of acid catalyst to a feed rate of hydrocarbon; an amount of fresh acid inventory; a feed rate of sulfur; a settler acid level; an alkylation reaction temperature; an acid boot valve position; a boiling end point of an alkylate; a mass of residue in an alkylate; a ratio of a flow rate of alkylate to a feed rate of olefin; an alkylate color; or a debutanizer acid boot level.
E. The method of any one of Paragraphs A-D, wherein determining the factor comprises computing a weighted combination of the differences between the one or more operating parameters and the one or more target operating parameters, the weighted combination comprising multiplying each difference by a respective weighting coefficient and summing resulting products or wherein applying a non-linear mapping function to at least one of the differences, the non-linear mapping function comprising a quadratic, exponential, logarithmic, or piecewise-linear function of the difference.
F. The method of any one of Paragraphs A-E, wherein determining the factor comprises assigning weighting coefficients to different ones of the one or more operating parameters, such that the factor is more sensitive to deviations in a first subset of the operating parameters than to deviations in a second subset of the operating parameters.
G. The method of Paragraph F, wherein determining the factor further comprises adjusting at least one weighting coefficient in response to one of the one or more operating parameters crossing a threshold value, such that the factor becomes more sensitive to the threshold-exceeding operating parameter.
H. The method of any one of Paragraphs A-G, wherein the one or more operating parameters further comprise a rate of change of at least one process variable selected from an alkylation reaction temperature, an acid purity, an isobutane recycle purity, or a sulfur content, and wherein the factor is based at least in part on the rate of change.
I. The method of any one of Paragraphs A-H, wherein determining the factor comprises increasing a persistence component associated with at least one of the one or more operating parameters when the at least one operating parameter remains outside a corresponding target range for more than a predetermined duration, and determining the factor based on both the difference and the persistence component.
J. The method of any one of Paragraphs A-I, further comprising detecting a faulty sensor based on a plausibility check of the measurements or a comparison between redundant sensors, and excluding a measurement from the faulty sensor from determining the factor.
K. The method of any one of Paragraphs A-J, wherein the one or more operating parameters further comprise at least one estimated parameter determined using a process model of the alkylation process, and wherein the factor is based at least in part on the estimated parameter.
L. A system comprising: a control circuit comprising one or more processors and memory structured to store instructions that, when executed by the one or more processors, cause the control circuit to: receive, by one or more processors and from one or more sensors, measurements indicative of one or more operating parameters of an alkylation process; determine, by the one or more processors, a difference between the one or more operating parameters of the alkylation process and one or more target operating parameters of the alkylation process; determine, by the one or more processors, a factor associated with one or more of the one or more operating parameters, wherein the factor is based on the difference; and provide, by the one or more processors, an indication of the factor to an operator of the alkylation process.
M. The system of Paragraph L, wherein the factor is a composite value determined according to two or more operating parameters.
N. The system of either one of Paragraphs L or M, wherein the control circuit is configured to change a position of a valve, a flow rate of isobutane, a flow rate of catalyst, a flow rate of olefin, or an operating temperature associated with the alkylation process in response to the factor.
O. The system of any one of Paragraphs L-N, wherein the control circuit is configured to determine the factor by computing a weighted combination of the differences between the one or more operating parameters and the one or more target operating parameters, the weighted combination comprising multiplying each difference by a respective weighting coefficient and summing resulting products.
P. The system of any one of Paragraphs L-O, wherein the control circuit is configured to determine a plurality of indices, including at least an acid health index and a product quality index, each based on a different subset of the one or more operating parameters, and to determine the factor according to the plurality of indices.
Q. The system of any one of Paragraphs L-P, wherein the control circuit is configured to store, in the memory, historical values of the one or more operating parameters, the factor, and one or more control actions, and to apply a predictive model trained on the historical values to estimate a future value of the factor or a probability of runaway acid conditions.
R. The system of any one of Paragraphs L-Q, wherein providing the indication of the factor to the operator of the alkylation process comprises displaying, on a graphical user interface, a breakdown of contributions of individual operating parameters to the factor.
S. A method of alkylating an olefin in the presence of an acid catalyst, the method comprising: receiving, by one or more processors and from one or more sensors, measurements indicative of two or more operating parameters of an alkylation system; determining, by the one or more processors, a difference between the two or more operating parameters of the alkylation system and corresponding target operating parameters of the alkylation system; determining, by the one or more processors, a factor associated with two or more of the one or more operating parameters, wherein the factor is based on the difference of the two or more factors and the corresponding target operating parameters; and changing a position of a valve, a flow rate of isobutane, a flow rate of catalyst, a flow rate of olefin, or an operating temperature associated with the alkylation system in response to the factor, wherein the one or more operating parameters of the alkylation system include at least two or more of: an acid purity; an isobutane recycle purity; a ratio of a feed rate of isobutane to a feed rate of olefin; a feed rate of isobutane to a catalyst regenerator; a ratio of a feed rate of acid catalyst to a feed rate of hydrocarbon; an amount of fresh acid inventory; a feed rate of sulfur; a settler acid level; an alkylation reaction temperature; an acid boot valve position; a boiling end point of an alkylate; a mass of residue in an alkylate; a ratio of a flow rate of alkylate to a feed rate of olefin; an alkylate color; or a debutanizer acid boot level.
T. The method of Paragraph S, further comprising displaying, by the one or more processors, the factor to an operator of the alkylation system.
U. The method of either one of Paragraphs S or T, wherein the receiving and determining steps are repeated periodically such that the factor is periodically updated.
V. The method of Paragraph U, wherein the factor is updated every 5 minutes or less.
W. The method of any one of Paragraphs S-V, wherein the changing step includes stopping a flow of olefin or stopping a flow of acid feed to the alkylation system when the factor exceeds a predetermined factor.
X. The method of any one of Paragraphs S-W, wherein determining the factor comprises computing a weighted combination of differences between the two or more operating parameters and the corresponding target operating parameters, and wherein at least one weighting coefficient is adjusted automatically in response to a change in a feed composition, an ambient condition, or a desired product specification.
Y. The method of any one of Paragraphs S-X, further comprising storing, in a computer memory, the factor and the measurements indicative of the two or more operating parameters, and using the stored factor and measurements to update a predictive model configured to estimate a future value of the factor or a probability of runaway acid conditions.
Z. The method of any one of Paragraphs S-Y, wherein the alkylation system comprises a plurality of alkylation units, the method further comprising determining a separate factor for each of the plurality of alkylation units and adjusting a distribution of olefin feed among the plurality of alkylation units in response to the separate factors.

Other embodiments are set forth in the following claims.

What is claimed is:

1. A method comprising:
receiving, by one or more processors and from one or more sensors, measurements indicative of one or more operating parameters of an alkylation process;
determining, by the one or more processors, a difference between the one or more operating parameters of the alkylation process and one or more target operating parameters of the alkylation process;
determining, by the one or more processors, a factor associated with one or more of the one or more operating parameters, wherein the factor is based on the difference; and
providing, by the one or more processors, an indication of the factor to an operator of the alkylation process.

2. The method of claim 1, wherein the factor is a composite value determined according to two or more operating parameters.

3. The method of claim 1, further comprising changing a position of a valve, a flow rate of isobutane, a flow rate of catalyst, a flow rate of olefin, or an operating temperature associated with the alkylation process in response to the factor.

4. The method of claim 1, wherein the one or more operating parameters of the alkylation process include at least one of:
an acid purity;
an isobutane recycle purity;
a ratio of a feed rate of isobutane to a feed rate of olefin;
a feed rate of isobutane to a catalyst regenerator;
a ratio of a feed rate of acid catalyst to a feed rate of hydrocarbon;
an amount of fresh acid inventory;
a feed rate of sulfur;
a settler acid level;
an alkylation reaction temperature;
an acid boot valve position;
a boiling end point of an alkylate;
a mass of residue in an alkylate;
a ratio of a flow rate of alkylate to a feed rate of olefin;
an alkylate color; or
a debutanizer acid boot level.

5. The method of claim 1, wherein determining the factor comprises computing a weighted combination of the differences between the one or more operating parameters and the one or more target operating parameters, the weighted combination comprising multiplying each difference by a respective weighting coefficient and summing resulting products or wherein applying a non-linear mapping function to at least one of the differences, the non-linear mapping function comprising a quadratic, exponential, logarithmic, or piecewise-linear function of the difference.

6. The method of claim 1, wherein determining the factor comprises assigning weighting coefficients to different ones of the one or more operating parameters, such that the factor is more sensitive to deviations in a first subset of the operating parameters than to deviations in a second subset of the operating parameters.

7. The method of claim 6, wherein determining the factor further comprises adjusting at least one weighting coefficient in response to one of the one or more operating parameters crossing a threshold value, such that the factor becomes more sensitive to the threshold-exceeding operating parameter.

8. A system comprising:
a control circuit comprising one or more processors and memory structured to store instructions that, when executed by the one or more processors, cause the control circuit to:
receive, by one or more processors and from one or more sensors, measurements indicative of one or more operating parameters of an alkylation process;
determine, by the one or more processors, a difference between the one or more operating parameters of the alkylation process and one or more target operating parameters of the alkylation process;
determine, by the one or more processors, a factor associated with one or more of the one or more operating parameters, wherein the factor is based on the difference; and
provide, by the one or more processors, an indication of the factor to an operator of the alkylation process.

9. The system of claim 8, wherein the factor is a composite value determined according to two or more operating parameters.

10. The system of claim 8, wherein the control circuit is configured to change a position of a valve, a flow rate of isobutane, a flow rate of catalyst, a flow rate of olefin, or an operating temperature associated with the alkylation process in response to the factor.

11. The system of claim 8 wherein the control circuit is configured to determine the factor by computing a weighted combination of the differences between the one or more operating parameters and the one or more target operating parameters, the weighted combination comprising multiplying each difference by a respective weighting coefficient and summing resulting products.

12. The system of claim 8, wherein the control circuit is configured to determine a plurality of indices, including at least an acid health index and a product quality index, each based on a different subset of the one or more operating parameters, and to determine the factor according to the plurality of indices.

13. The system of claim 8, wherein the control circuit is configured to store, in the memory, historical values of the one or more operating parameters, the factor, and one or more control actions, and to apply a predictive model trained on the historical values to estimate a future value of the factor or a probability of runaway acid conditions.

14. A method of alkylating an olefin in the presence of an acid catalyst, the method comprising:
receiving, by one or more processors and from one or more sensors, measurements indicative of two or more operating parameters of an alkylation system;
determining, by the one or more processors, a difference between the two or more operating parameters of the alkylation system and corresponding target operating parameters of the alkylation system;
determining, by the one or more processors, a factor associated with two or more of the one or more operating parameters, wherein the factor is based on the difference of the two or more factors and the corresponding target operating parameters; and
changing a position of a valve, a flow rate of isobutane, a flow rate of catalyst, a flow rate of olefin, or an operating temperature associated with the alkylation system in response to the factor,
wherein the one or more operating parameters of the alkylation system include at least two or more of:
an acid purity;
an isobutane recycle purity;
a ratio of a feed rate of isobutane to a feed rate of olefin;
a feed rate of isobutane to a catalyst regenerator;
a ratio of a feed rate of acid catalyst to a feed rate of hydrocarbon;
an amount of fresh acid inventory;
a feed rate of sulfur;
a settler acid level;
an alkylation reaction temperature;
an acid boot valve position;
a boiling end point of an alkylate;
a mass of residue in an alkylate;
a ratio of a flow rate of alkylate to a feed rate of olefin;
an alkylate color; or
a debutanizer acid boot level.

15. The method of claim 14, further comprising displaying, by the one or more processors, the factor to an operator of the alkylation system.

16. The method of claim 14, wherein the receiving and determining steps are repeated periodically such that the factor is periodically updated.

17. The method of claim 16, wherein the factor is updated every 5 minutes or less.

18. The method of claim 14, wherein the changing step includes stopping a flow of olefin or stopping a flow of acid feed to the alkylation system when the factor exceeds a predetermined factor.

19. The method of claim 14, wherein determining the factor comprises computing a weighted combination of differences between the two or more operating parameters and the corresponding target operating parameters, and wherein at least one weighting coefficient is adjusted automatically in response to a change in a feed composition, an ambient condition, or a desired product specification.

20. The method of claim 14, wherein the alkylation system comprises a plurality of alkylation units, the method further comprising determining a separate factor for each of the plurality of alkylation units and adjusting a distribution of olefin feed among the plurality of alkylation units in response to the separate factors.
